(19) United States
(12) Patent Application Publication
(10) Pub. No.: US 2020/0206095 A1
(43) Pub. Date: Jul. 2, 2020

(54) DEODORANT COMPOSITION

(71) Applicant: FUJIFILM Corporation, Tokyo (JP)

(72) Inventor: Kazunori TAKAHASHI, Kanagawa (JP)

(73) Assignee: FUJIFILM Corporation, Tokyo (JP)

(21) Appl. No.: 16/816,417

(22) Filed: Mar. 12, 2020

Related U.S. Application Data
(63) Continuation of application No. PCT/JP2018/032924, filed on Sep. 5, 2018.

(30) Foreign Application Priority Data
Sep. 29, 2017 (JP) 2017-189852

(51) Int. Cl. A61K 8/02; A61K 8/27; A61Q 15/00

(52) U.S. Cl. CPC A61K 8/0241; A61K 2800/651; A61Q 15/00; A61K 8/27

(57) ABSTRACT

Provided a deodorant composition including dispersed particles which have an average secondary particle diameter of 200 nm or less and are formed of at least one kind of particles selected from metal particles or metal oxide particles and each of which has a surface that does not contain a dispersant; and at least one kind selected from an aqueous solvent other than water or a metal salt having a monovalent, a divalent, or a trivalent metal ion.

### CROSS-REFERENCE TO RELATED APPLICATIONS

This application is a continuation application of International Application No. PCT/JP2018/032924, filed Sep. 5, 2018, the disclosure of which is incorporated herein by reference in its entirety. Further, this application claims priority from Japanese Patent Application No. 2017-189852, filed Sep. 29, 2017, the disclosures of which is incorporated herein by reference in its entirety.

### BACKGROUND OF THE INVENTION

### 1. Field of the Invention

The present disclosure relates to a deodorant composition.

### 2. Description of the Related Art

In the related art, various techniques related to deodorizing performance with respect to an odor material have been suggested.

As a composition having excellent an adsorption effect on both an amine-based odor component and a sulfur-containing odor component, JP2009-227991A describes an adsorptive composition which includes at least one kind from among fatty acid metal salts such as Ni, Cu, and Co, and a composition containing ultrafine metal particles having plasmon absorption at 300 to 700 nm.

JP2014-183962A describes a deodorant which contains silicon dioxide and zinc oxide as main components, and a metal hydroxide or a metal oxide acting as a zinc component elution inhibitor.

Further, since the characteristics of metal oxide particles having a particle diameter of nanometers are greatly different from typical metal oxide particles, and particularly the surface activity and the surface area are large, use of the metal oxide particles have been suggested in the fields of catalysts, adsorbents, and the like.

JP2016-160124A describes a method of producing nanometer-sized copper oxide fine particles by merging and reacting a copper (II) salt solution with a basic compound solution using a flow type reaction (also referred to as a flow reactor).

### SUMMARY OF THE INVENTION

As described above, due to the large surface activity and the surface area of the nanometer-sized particles which are metal particles or metal oxide particles, use of these particles in various fields such as catalysts and adsorbents. As an aspect of application of such nanometer-sized particles, a deodorant composition is exemplified.

Further, according to the examination of the present inventors, it was found that a deodorant composition containing nanometer-sized particles which are metal particles or metal oxide particles exhibits an excellent deodorizing effect, but the deodorizing effect decreases as the deodorant composition is stored and aged. The decrease of the deodorizing effect with time is significant particularly in a case where the content of the particles is low (for example, the content of the particles is 0.1% by mass or less).

An object of an embodiment of the present invention is to provide a deodorant composition in which a decrease in the deodorizing effect is suppressed in a case of storage for a long period of time.

Specific means for achieving the above-described object includes the following aspects.

<1> A deodorant composition comprising: dispersed particles which have an average secondary particle diameter of 200 nm or less and are formed of at least one kind of particles selected from metal particles or metal oxide particles and each of which has a surface that does not contain a dispersant; and at least one kind selected from an aqueous solvent other than water or a metal salt having a monovalent, a divalent, or a trivalent metal ion.

<2> The deodorant composition according to <1>, in which the metal salt is at least one kind selected from metal salts having divalent metal ions.

<3> The deodorant composition according to <2>, in which the metal salt is at least one kind selected from a copper salt, a zinc salt, or a magnesium salt.

<4> The deodorant composition according to any one of <1> to <3>, in which a content of the metal ion derived from the metal salt is in a range of 10% by mass to 50% by mass with respect to a total mass of the dispersed particles contained in the deodorant composition.

<5> The deodorant composition according to any one of <1> to <4>, in which the aqueous solvent is alcohol, and a content of the alcohol is 20% by mass or greater with respect to a total mass of a dispersion medium contained in the deodorant composition.

<6> The deodorant composition according to any one of <1> to <5>, in which the aqueous solvent is monovalent alcohol having 1 to 3 carbon atoms.

<7> The deodorant composition according to any one of <1> to <6>, in which a molar ratio of the dispersed particles to hydroxide ions contained in the deodorant composition is 800 or greater.

<8> The deodorant composition according to any one of <1> to <7>, in which the dispersed particles are copper oxide particles.

<9> The deodorant composition according to any one of <1> to <8>, in which a content of the dispersed particles is in a range of 0.0001% by mass to 14% by mass with respect to a total mass of the deodorant composition.

<10> The deodorant composition according to any one of <1> to <9>, which is used for deodorizing odor caused by hydrogen sulfide.

According to the embodiment of the present invention, it is possible to provide a deodorant composition in which a decrease in the deodorizing effect is suppressed in a case of storage for a long period of time.

### BRIEF DESCRIPTION OF THE DRAWINGS

FIG. 1 is a flow diagram illustrating a preferred embodiment of a method of producing dispersed particles according to the present disclosure.

FIG. 2 shows XPS measurement data for dispersed particles 1 prepared in examples and is related to carbon C1s.

FIG. 3 shows XPS measurement data for dispersed particles 1 obtained in examples and is related to copper Cu₂p_{3/2}.

FIG. 4 shows an area A₁ of a peak derived from divalent CuO in the XPS measurement data and an area A₂ of a peak derived from all components containing Cu.

### DESCRIPTION OF THE PREFERRED EMBODIMENTS

Hereinafter, a deodorant composition according to an embodiment of the present disclosure will be described. Here, the deodorant composition according to the embodiment of the present disclosure is not limited to the embodiment described below, and changes can be made as appropriate within the range of the purpose according to the present disclosure.

The numerical ranges shown using “to” in the present disclosure indicate ranges including the numerical values described before and after “to” as the lower limits and the upper limits.

In the numerical ranges described in a stepwise manner in the present disclosure, the upper limits or the lower limits described in certain numerical ranges may be replaced with the upper limits or the lower limits in other numerical ranges described in a stepwise manner.

Further, in the numerical ranges described in the present disclosure, the upper limits or the lower limits described in certain numerical ranges may be replaced with values described in examples.

In the present disclosure, in a case where a plurality of substances corresponding to respective components in a composition are present, the amount of the respective components in the composition indicates the total amount of the plurality of substances present in the composition unless otherwise specified.

In the present disclosure, a combination of two or more preferable aspects is a more preferable aspect.

In the present disclosure, the term “copper salt” which is simply described indicates a “copper (II) salt” unless otherwise specified.

[Deodorant Composition]

A deodorant composition according to the embodiment of the present disclosure contains dispersed particles (hereinafter, also referred to as specific dispersed particles) which have an average secondary particle diameter of 200 nm or less and are formed of at least one kind of particles selected from metal particles or metal oxide particles and each of which has a surface that does not contain a dispersant; and at least one kind selected from an aqueous solvent other than water (hereinafter, also referred to as a specific solvent”) or a metal salt having a monovalent, a divalent, or a trivalent metal ion (hereinafter, also referred to as a specific metal salt).

The deodorant composition according to the embodiment of the present disclosure has a form of a dispersion liquid.

Even in a case where the deodorant composition according to the embodiment of the present disclosure is stored for a long period of time, a decrease in the deodorizing effect is suppressed.

Here, the storage for a long period of time in the present disclosure indicates a storage period of 3 months or longer.

In the present disclosure, the determination of whether the deodorizing effect is decreased at the time of storage of the deodorant composition is made based on the comparison between the deodorizing effects before and after the storage of the deodorant composition sealed in a storage container for a predetermined period of time under a temperature condition of 25° C. Further, a specific method of confirming the deodorizing effect will be described below.

Further, the deodorant composition according to the embodiment of the present disclosure can be stored without particular limitation on the storage location as long as the environment is typically applied to the storage of the deodorant composition.

According to the present inventors, the reason why the deodorant composition according to the embodiment of the present disclosure exhibits the above-described effect is assumed as follows. However, the following assumption does not provide limitative interpretation of the effects of the deodorant composition according to the embodiment of the present disclosure but the description of an example.

In the deodorant composition according to the embodiment of the present disclosure, the specific dispersed particles which are components exhibiting the deodorizing effect are present in an unstable state in the system as compared to dispersed particles which are dispersed by a dispersant present on the surface of each particle. Accordingly, it is considered that the deodorizing effect of the deodorant composition is decreased due to change of the surface property of the specific dispersed particles with time.

On the contrary, it is assumed that since the deodorant composition according to the embodiment of the present disclosure contains at least one selected from a specific solvent or a specific metal salt, a change of the surface property of the specific dispersed particles with time is suppressed so that a decrease in the deodorizing effect is effectively suppressed.

A decrease in the deodorizing effect is particularly significant in a case where the content of the specific dispersed particles in the deodorant composition containing the specific dispersed particles is low. However, in a case of the deodorant composition according to the embodiment of the present disclosure, a decrease in the deodorizing effect is remarkably suppressed even in the above-described case.

According to the examination conducted by the present inventors, as one hypothesis which can explain the decrease in the deodorizing effect occurring at the time of allowing the deodorant composition containing the specific dispersed particles to be aged, it is considered that the hydroxide ion (OH⁻) contained in the composition acts on the specific dispersed particles so that the element which is present on the surface of each specific dispersed particle or in the vicinity of the surface thereof and contributes to the dispersion stability is replaced with the hydroxide ion with time. For example, as the element contributing to the dispersion stability of the specific dispersed particles in a case of the deodorant composition described in the examples below, an acetate ion present on the surface of a copper oxide particle which is an aspect of the specific dispersed particle or in the vicinity of the surface thereof is exemplified.

On the contrary, it is assumed that since the deodorant composition according to the embodiment of the present disclosure contains at least one selected from a specific solvent or a specific metal salt, the amount of the hydroxide ions in the composition is decreased, and thus the action of the hydroxide ions on the specific dispersed particles can be suppressed.

Meanwhile, in the adsorptive composition described in Patent Document 1 (JP2009-227991A) and the deodorant described in Patent Document 2 (JP2014-183962A), the decrease in the deodorizing effect with time has not been paid attention to. Further, the cupric oxide fine particles obtained in Patent Document 3 (JP2016-160124A) is not used in the deodorant composition.

Hereinafter, each component in the deodorant composition according to the embodiment of the present disclosure will be described in detail.

(Specific Dispersed Particles)

The deodorant composition according to the embodiment of the present disclosure contains dispersed particles (specific dispersed particles) which have an average secondary particle diameter of 200 nm or less and contain a metal or a metal oxide and each of which has a surface that does not contain a dispersant.

The deodorant composition may contain only one or two or more kinds of specific dispersed particles.

In the present disclosure, the “dispersant” indicates known dispersants roughly classified into polymer dispersants such as dispersion resins and low-molecular-weight dispersants such as surfactants.

Further, in the present disclosure, the expression “specific dispersed particles each have a surface that does not contain a dispersant” means that a dispersant is not present, on the surface or in the vicinity of the surface of the specific dispersed particles, in an amount that enables dispersion of the specific dispersed particles in the deodorant composition.

In other words, the deodorant composition according to the embodiment of the present disclosure does not contain a dispersant or does not substantially contain a dispersant. Here, the expression of “does not substantially contain a dispersant” means that the content of the dispersant is less than 0.0001% by mass with respect to the total amount of the dispersed particles containing specific dispersed particles.

The specific dispersed particles have an average secondary particle diameter of 200 nm or less, preferably 100 nm or less, more preferably 20 nm to 50 nm, and still more preferably 20 nm to 30 nm. In a case where the average secondary particle diameter of the specific dispersed particles is 200 nm or less, the dispersibility is excellent, and a high deodorizing effect is obtained.

The secondary particles in the present disclosure are defined as an aggregate formed by the primary particles being fused or brought into contact with one another. The average secondary particle diameter is a value obtained by measuring the diameter of each secondary particle from an image of an electron microscope and averaging the diameters of 90% of secondary particles obtained by removing 5% of particles on a side where the diameters thereof are the smallest and 5% of particles on a side where the diameters thereof are the largest from among the total number of secondary particles.

Here, the diameter indicates a circumscribed circle equivalent diameter of each secondary particle.

From the viewpoint of the deodorizing effect, the specific dispersed particles have an average primary particle diameter of 5 nm to 20 nm, preferably 5 nm to 15 nm, and more preferably 5 nm to 10 nm.

The average primary particle diameter is a value obtained by measuring the diameter of each primary particle from an image of an electron microscope and averaging the diameters of 90% of primary particles obtained by removing 5% of primary particles on a side where the diameters thereof are the smallest and 5% of primary particles on a side where the diameters thereof are the largest from among the total number of primary particles. Here, the diameter indicates a circumscribed circle equivalent diameter of each primary particle.

The average particle diameter of the specific dispersed particles can be measured by dynamic light scattering method with a particle size distribution measuring machine that performs measurement using laser diffraction. Specifically, the measurement can be performed according to the method described in examples.

The shape of the specific dispersed particles is not particularly limited as long as the particles are particulate. The term “particulate” indicates a small particle shape, and specific examples thereof include a spherical shape, an ellipsoidal shape, a rod shape, and a plate shape. The specific dispersed particles do not necessarily have a perfect spherical or ellipsoidal shape, and a part thereof may be distorted. It is advantageous that the specific dispersed particles have a spherical shape rather than a rod shape or a plate shape from the viewpoint that the contact area between particles is decreased so that aggregation is unlikely to occur.

The specific surface area of the specific dispersed particles is preferably 10 m²/g or greater. The absolute value of the zeta potential of the specific dispersed particles is preferably 10 mV or greater. Further, it is more preferable that the specific surface area of the specific dispersed particles is 10 m²/g or greater and the absolute value of the zeta potential of the specific dispersed particles is 10 mV or greater. The items related to the measurement of the specific surface area and the zeta potential will be described in the section of “copper oxide particles A” described below as one preferable aspect of the specific dispersed particles.

In a case where the specific dispersed particles are metal particles, the metal particles can be selected from the metal particles exhibiting the deodorizing effect. From the viewpoint of the deodorizing effect, metal particles containing at least one element selected from the element group consisting of Au, Ag, Pd, Pt, Cu, Zn, Fe, Ni, Mg, and Zr are preferable, and metal particles containing Cu, Zn, Ag, or Mg are more preferable.

In a case where the specific dispersed particles are metal oxide particles, the metal oxide particles can be selected from the metal oxide particles exhibiting the deodorizing effect. From the viewpoint of the deodorizing effect, at least one kind of metal oxide particles selected from the group consisting of copper oxide, zinc oxide, and magnesium oxide are preferable, and at least one kind of metal oxide particles selected from copper oxide and zinc oxide are preferable. From the viewpoints of the deodorizing effect with respect to hydrogen sulfide and the production suitability, copper oxide particles are particularly preferable as the metal oxide particles.

As a suitable aspect of the copper oxide particles which are specific dispersed particles, copper oxide particles described in JP2016-160124A are exemplified, and copper oxide particles obtained according to the production method described in the same document can be used as the specific dispersed particles in the present disclosure.

One suitable aspect of the specific dispersed particles relates to copper oxide particles, in which each surface of a copper oxide particle a is covered with a coating layer b containing a monovalent copper compound. Hereinafter, the copper oxide particles according to this aspect will be described by being referred to as “copper oxide particles A”.

It is preferable that the specific surface area of the copper oxide particles A is 100 m²/g or greater, the average primary particle diameter thereof is in a range of 5 nm to 20 nm, and the average secondary particle diameter thereof is in a range of 5 nm to 50 nm.

As the copper oxide particles a, copper oxide particles formed of an oxide of monovalent or divalent copper containing copper (I) oxide particles (Cu₂O particles) as a main component are preferable, and copper (I) oxide particles (Cu₂O particles), copper (II) oxide particles (CuO particles), or a mixture of copper (I) oxide particles and copper (II) oxide particles is more preferable. Here, the main component indicates that the amount thereof is in a range of 50% mass to 100% by mass, preferably in a range of 70% mass to 100% by mass, and more preferably in a range of 85% mass to 100% by mass of the copper oxide particles a.

The surface of each copper oxide particle a is covered with the coating layer b. A part or the entirety of the surface of each copper oxide particle a may be covered with the coating layer b.

The surface of the copper oxide particle a being covered with the coating layer b containing a monovalent copper compound can be confirmed from the presence of a peak derived from divalent CuO in a range of 938.5 eV to 948 eV in the Cu₂p_{3/2} spectrum according to X-ray photoelectron spectroscopy (XPS).

The coating layer b contains a monovalent copper compound and it is preferable that the coating layer b contains a monovalent copper compound as a main component. As the monovalent copper compound, cuprous oxide is preferable. Here, the main component indicates that the amount thereof is in a range of 30% mass to 100% by mass, preferably in a range of 50% mass to 100% by mass, more preferably in a range of 70% mass to 100% by mass, and most preferably in a range of 85% mass to 100% by mass of the coating layer b. The coating layer b may contain copper hydroxide, salts derived from raw materials, and the like as components other than the monovalent copper compound.

It is preferable that the copper oxide particle a is protected by an organic layer c derived from acetic acid or an acetate and more preferable that the surface of the coating layer b containing a monovalent copper compound is covered with the organic layer c derived from acetic acid or an acetate. In this manner, in a case where the copper oxide particles A are dispersed in a dispersion medium, since the particles repel one another due to the charge protected by the organic layer c derived from acetic acid or an acetate, aggregation is suppressed, copper oxide particles A are stabilized without precipitation even in a case where a dispersant is not added or a dispersion treatment is not performed so that the dispersibility becomes excellent.

It is preferable that the organic layer c is a layer containing an organic substance, as a main component, derived from acetic acid or an acetate. Here, the main component indicates that the amount thereof is in a range of 30% mass to 100% by mass, preferably in a range of 50% mass to 100% by mass, more preferably in a range of 70% mass to 100% by mass, and most preferably in a range of 85% mass to 100% by mass of the organic layer c. Examples of the organic substance derived from acetic acid or an acetate include sodium acetate and lithium acetate. Further, the organic layer c may contain copper salts derived from raw materials as components other than the organic substance derived from acetic acid or an acetate.

The presence of the organic layer c can be confirmed by time-of-flight secondary ion mass spectrometry (TOF-SIMS).

In the copper oxide particles A, it is preferable that a peak area ratio (1) in X-ray diffraction using CuKα as an X-ray source is in a range of 0.01 to 0.10.

Peak area ratio (1)=A₁/(A₁+A₂)

A₁: a peak area of a peak derived from divalent CuO which is present in a range of 938.5 eV to 948 eV

A₂: a peak area of a peak derived from all components containing Cu which is present in a range of 928 eV to 938.5 eV

In a case where the peak area ratio (1) is set to be in the above-described range, there is an advantage that each copper oxide particle A is likely to be covered with the organic layer c derived from acetic acid or an acetate and the copper oxide particles A can be stably dispersed in a dispersion medium.

The specific surface area of the copper oxide particles A is preferably 100 m²/g or greater and more preferably in a range of 100 m²/g to 250 m²/g. In a case where the specific surface area thereof is in the above-described range, the deodorizing effect of the copper oxide particles A is excellent. The specific surface area can be measured according to the BET one-point method.

The shape of the copper oxide particles A is the same as the shape of the specific dispersed particles.

It is preferable that the copper oxide particles A have a zeta potential of 30 mV to 50 mV in a case of being dispersed in water having a pH of 6.8. In a case where the zeta potential is in the above-described range, the dispersibility becomes excellent so that aggregation of the particles in the dispersion liquid is suppressed. Therefore, copper oxide particles A having a desired particle diameter can be obtained. The zeta potential can be measured according to a known method.

A method of producing the copper oxide particles A is not particularly limited, but it is preferable that the copper oxide particles A are continuously produced by a flow type reaction.

The deodorant composition according to the embodiment of the present disclosure may contain dispersed particles other than the specific dispersed particles.

In a case where the deodorant composition contains dispersed particles other than the specific dispersed particles, from the viewpoints of the deodorizing effect and suppressing a decrease in the deodorizing effect in a case where the deodorant composition is stored for a long period of time, the content of the specific dispersed particles is preferably 10% by mass or greater, more preferably 30% by mass or greater, and still more preferably 50% by mass or greater with respect to the total amount of the dispersed particles.

All the dispersed particles in the deodorant composition according to the embodiment of the present disclosure may be specific dispersed particles (that is, 100% by mass).

The content of the specific dispersed particles in the deodorant composition according to the embodiment of the present disclosure is not particularly limited as long as the deodorizing effect is obtained.

The content of the specific dispersed particles is preferably 50% by mass or less with respect to the total mass of the deodorant composition.

In the deodorant composition according to the embodiment of the present disclosure, a decrease in the deodorizing effect at the time of storage for a long period of time is suppressed even in a case where the specific dispersed particles have a low concentration.

In a case where the specific dispersed particles have a low concentration, the content of the specific dispersed particles in the deodorant composition is preferably in a range of 0.0001% by mass to 14% by mass, more preferably in a range of 0.0001% by mass to 10% by mass, still more preferably in a range of 0.0005% by mass to 5% by mass, and particularly preferably in a range of 0.0005% by mass to 1% by mass with respect to the total mass of the deodorant composition.

In a case where the hydroxide ions are present in the deodorant composition in an amount that enables measurement, the molar ratio of the specific dispersed particles to the hydroxide ions contained in the deodorant composition is preferably 800 or greater.

In a case where the deodorant composition contains specific metal salts and does not contain specific solvents, the molar ratio of the specific dispersed particles to the hydroxide ions is preferably 9000 or greater, more preferably 11000 or greater, and still more preferably 13000 or greater.

In a case where the deodorant composition contains specific solvents and does not contain specific metal salts, the molar ratio of the specific dispersed particles to the hydroxide ions is preferably 800 or greater, more preferably 850 or greater, and still more preferably 900 or greater.

In a case where the deodorant composition contains both specific metal salts and specific solvents, the molar ratio of the specific dispersed particles to the hydroxide ions is preferably 800 or greater, more preferably 850 or greater, and still more preferably 900 or greater.

In a case where the molar ratio of the specific dispersed particles to the hydroxide ions is in the above-described range, a decrease in the deodorizing effect at the time of storage for a long period of time is more effectively suppressed.

In the present disclosure, the molar ratio (x/y) of the specific dispersed particles (x) to the hydroxide ions (y) contained in the deodorant composition can be confirmed from the “concentration (mol/L) of specific particles” and the “concentration (mol/L)” of hydroxide ions which are obtained by performing measurement according to the following method. Further, both the “concentration (mol/L) of specific particles” and the concentration (mol/L) of hydroxide ions” are confirmed at 25° C.

Measurement of Concentration (mol/L) of Specific Particles

As the concentration (mol/L) of the specific dispersed particles, a measured value obtained by performing measurement using inductively coupled plasma (ICP) emission spectroscopy is used.

The concentration of the specific particles in the present specification is a value measured using an ICP emission spectrometer (PS3520VDDII, manufactured by Hitachi High-Tech Science Corporation).

Measurement of Concentration (mol/L) of Hydroxide Ions

The concentration (mol/L) of the hydroxide ions contained in the deodorant composition can be measured using the following method (1), (2), or (3) according to the aspect of the deodorant composition.

(1) Case where Deodorant Composition Contains Specific Metal Salt and Does Not Contain Specific Solvent

The concentration (mol/L) of the hydroxide ions is calculated from the measured value obtained by measuring the pH of the deodorant composition using a pH meter and is set as the concentration (mol/L) of the hydroxide ions in the deodorant composition.

(2) Case where Deodorant Composition Contains Specific Solvent and Does Not Contain Specific Metal Salt

The concentration of water in the deodorant composition is measured, and the concentration (mol/L) of the hydroxide ions in the deodorant composition is calculated from the obtained concentration of water. Specifically, the concentration of water of the liquid which becomes a dispersion medium of the dispersed particles in the deodorant composition is measured using a Karl Fischer moisture measuring device. Next, the concentration (mol/L) of the hydroxide ions in the deodorant composition is calculated from the volume ratio of water in the dispersion medium calculated from the measurement results using the above-described measuring device by assuming that the concentration of the hydroxide ions of water is 1×10⁻⁷.

Specifically, as the Karl Fischer moisture measuring device, for example, a device “851 Titrando” (manufactured by Metrohm AG) can be used.

In a case where water used for preparation of the deodorant composition can be specified, the pH of water used for preparation of the deodorant composition is measured using a pH meter, the concentration (mol/L) of the hydroxide ions is calculated from the obtained results, and the concentration (mol/L) of the hydroxide ions in the deodorant composition can be determined from the ratio of the amount of the specific solvent to the total amount of the deodorant composition.

(3) Case where Deodorant Composition Contains Both Specific Metal Salt and Specific Solvent

The amount of the specific metal salt and the concentration of water contained in the deodorant composition are measured, and the concentration (mol/L) of the hydroxide ions in the deodorant composition is calculated based on the obtained amount of the specific metal salt and concentration of water. Specifically, first, the amount of the specific metal salt is measured according to the ion chromatography. The concentration of water in the deodorant composition is separately measured in the same manner as in (2) described above according to the Karl Fischer method. A specific metal salt aqueous solution is prepared based on the obtained amount of the specific metal salt and concentration of water. The concentration (mol/L) of the hydroxide ions in the specific metal salt aqueous solution is calculated by measuring the pH of the prepared specific metal salt aqueous solution using a pH meter. The concentration (mol/L) of the hydroxide ions in the deodorant composition is calculated based on the obtained concentration (mol/L) of the hydroxide ions in the specific metal salt aqueous solution and the volume ratio of water in the deodorant composition calculated from the concentration of water measured in the above-described manner.

Specifically, as the ion chromatography device, for example, “HIC-SP” (manufactured by Shimadzu Corporation) can be used.

Further, in a case where the specific metal salt solution water used for preparation of the deodorant composition can be specified, the concentration (mol/L) of the hydroxide ions is calculated from the pH of the specific metal salt aqueous solution, and the concentration (mol/L) of the hydroxide ions in the deodorant composition can be determined from the ratio of the amount of the specific solvent to the total amount of the deodorant composition.

Further, in a preparation example of obtaining a deodorant composition in which the content of alcohol is 10% by mass using copper oxide particles as the specific dispersed particles and alcohol as the specific solvent and using a 5 mass% aqueous solution of the specific metal salt, the fact that the sum of the molar ratios calculated using the following (1) and (2) is 800 or greater can be used as an indicator that the deodorant composition is satisfactorily stored for a long period of time due to the suppression of a decrease in the deodorizing effect.

(1): The pH of the 5 mass% aqueous solution of the specific metal salt used for preparation of the composition is measured to calculate the concentration of the hydroxide ions [OH⁻], and [CuO]/[OH⁻]×(8/90) is calculated from the separated measured concentration of copper oxide (CuO) particles.

(2): The concentration of the hydroxide ions [OH⁻] in the deodorant composition in which the content of alcohol is 10% by mass is calculated, and [CuO]/[OH⁻] is calculated from the separately measured concentration of copper oxide [CuO] particles.

(Specific Solvent and Specific Metal Salt)

The deodorant composition according to the embodiment of the present disclosure contains an aqueous solvent (specific solvent) other than water and at least one selected from metal salts (specific metal salts) having monovalent, divalent, or trivalent metal ions.

The deodorant composition may contain only the specific solvent, only the specific metal salt, or both the specific solvent and the specific metal salt.

The deodorant composition may contain only one or two or more kinds of specific metal salts.

Further, the deodorant composition may contain only one or two or more kinds of specific solvents. In a case where two or more kinds of specific solvents are selected, solvents which are compatible with each other may be selected.

<Specific Metal Salt>

The specific metal salt is a metal salt having a monovalent, divalent, or trivalent metal ion.

From the viewpoint of suppressing the decrease in the deodorizing effect at the time of storage for a long period of time, the metal ion contained in the specific metal salt is preferably monovalent or divalent and more preferably divalent.

The ion chromatography may be used to confirm whether the deodorant composition contains the specific metal salt. During the confirmation, a filtrate obtained by concentrating the target deodorant composition to have a concentration that enables measurement through ultrafiltration may be used as a sample. The above-described device can be used as the ion chromatography device.

Among specific metal salts, examples of the metal salt having a monovalent metal ion include alkali metal salts such as a Na salt, a K salt, and Li salt.

Among specific metal salts, examples of the metal salt having a divalent metal ion include a copper salt, a zinc salt, a magnesium salt, an iron salt, and a zirconium salt. From the viewpoint of suppressing a decrease in the deodorizing effect at the time of storage for a long period of time, at least one selected from a copper salt, a zinc salt, or a magnesium salt is preferable, and a copper salt or a zinc salt is more preferable.

Examples of the copper salt include copper (II) nitrate, copper (II) chloride, copper (II) bromide, copper (II) iodide, copper (II) sulfate, copper (II) formate, copper (II) acetate, copper (II) propionate, copper (II) isobutyrate, copper (II) oleate, copper (II) citrate, copper (II) phthalate, copper (II) oxalate, copper (II) tartrate, basic copper carbonate, basic copper sulfate, and hydrates of these copper salts, an inorganic compound complex of copper (such as a tetraamine copper complex), and an organic compound complex of copper (such as copper acetylacetonate). Among these, copper (II) nitrate or copper (II) acetate is preferable.

Examples of the zinc salt include zinc (II) acetate, zinc (II) sulfate, zinc (II) nitrate, and zinc (II) chloride. Among these, zinc (II) acetate or zinc (II) sulfate is preferable.

Examples of the magnesium salt include magnesium (II) acetate, magnesium (II) sulfate, magnesium (II) nitrate, magnesium (II) nitrate, magnesium (II) phosphate, and magnesium (II) chloride. Among these, magnesium (II) acetate or magnesium (II) nitrate is preferable.

From the viewpoint of suppressing the decrease in the deodorizing effect at the time of storage for a long period of time, the content of the metal ion derived from the metal salt in the deodorant composition is preferably in a range of 10% by mass to 50% by mass and more preferably in a range of 20% by mass to 50% by mass with respect to the total mass of the specific dispersed particles. In a case where the content of the metal ion is 20% by mass or greater, the effect of suppressing the decrease in the deodorizing effect is further improved. Further, in a case where the content thereof is 50% by mass or less, the effect of suppressing aggregation of the specific dispersed particles is improved, which is preferable.

The content of the metal ions in the deodorant composition can be confirmed according to the ion chromatography. The above-described device can be used as the ion chromatography device.

<Specific Solvent>

The specific solvent is an aqueous solvent other than water.

The deodorant composition may contain only one or two or more kinds of specific solvents.

The aqueous solvent other than water in the present disclosure indicates a solvent in which 5 g or more of a substance is dissolved in 100 g of water at a liquid temperature of 25° C.

In a case where the deodorant composition according to the embodiment of the present disclosure contains the specific solvent, the specific solvent may be contained in the deodorant composition as a part or the entirety of the dispersion medium contained in the deodorant composition.

As the specific solvent, a water-soluble organic solvent is preferable. Specific examples thereof include an alcohol, an ether, and a ketone. Among these, from the viewpoint of the effect of decreasing the amount of the hydroxide ions in the deodorant composition, the alcohol is preferable. As the alcohol, a monovalent alcohol having 1 to 3 carbon atoms is preferable.

Specific examples of the alcohol include methanol, ethanol, isopropanol, and normal propanol. Among these, from the viewpoint of the load on the environment and the human body, ethanol or isopropanol is more preferable.

Examples of the ketone include acetone and methyl ethyl ketone. Among these, acetone is preferable.

Examples of the ether include ethyl methyl ether and diethyl ether. Among these, diethyl ether is preferable.

In a case where the specific solvent is alcohol, from the viewpoint of suppressing a decrease in the deodorizing effect at the time of storage for a long period of time, the content of the alcohol in the deodorant composition is preferably 20% by mass or greater and more preferably 30% by mass or greater with respect to the total mass of the dispersion medium contained in the deodorant composition. It is preferable that the content of the alcohol is 20% by mass or greater because the effect of suppressing a decrease in the deodorizing effect is further improved. The upper limit of the alcohol content is not particularly limited, but may be 100% by mass with respect to the total mass of the dispersion medium contained in the deodorant composition. That is, the dispersion medium may be entirely formed of alcohol.

(Dispersion Medium)

The deodorant composition according to the embodiment of the present disclosure has a form of a dispersion liquid and contains a dispersion medium.

In a case where the deodorant composition contains a specific solvent, a part or the entirety of the dispersion medium may be the specific solvent.

The deodorant composition may contain water as the dispersion medium. The dispersion medium may be a mixture of water and the specific solvent or may be formed of only water. For example, in a case where the deodorant composition does not contain the specific solvent and contains only the specific metal salt, the dispersion medium in the deodorant composition can be formed of only water.

(Additive)

The deodorant composition according to the embodiment of the present disclosure may contain an additive as necessary. Examples of the additives include known additives such as an ultraviolet absorbing agent, a preservative, a pH adjuster, an antifoaming agent, and a dispersion stabilizer.

[Applications and Use Form of Deodorant Composition]

Examples of the odor component to be deodorized by the deodorant composition according to the embodiment of the present disclosure include hydrogen sulfide (H₂S) and methyl mercaptan (CH₃SH). The deodorant composition according to the embodiment of the present disclosure is particularly suitable for deodorizing hydrogen sulfide.

The use form of the deodorant composition according to the embodiment of the present disclosure is not particularly limited, and examples thereof include an aspect in which the deodorant composition is accommodated in a container and sprayed to a space or a target to be deodorized and an aspect in which a target to be deodorized is immersed in the deodorant composition.

[Production of Deodorant Composition]

A method of producing the deodorant composition according to the embodiment of the present disclosure is not particularly limited, and examples thereof include the following aspects (1) and (2).

1) An aspect in which a dispersion liquid containing specific dispersed particles is produced, the specific dispersed particles are filtrated, and a specific solvent and/or a solution containing a specific metal salt is added to the filtered specific dispersed particles; and

2) An aspect in which a dispersion liquid containing specific dispersed particles is produced, the dispersion liquid is concentrated by ultrafiltration, and a specific solvent and/or a solution containing a specific metal salt is added to the obtained concentrate

From the viewpoint of the production suitability, it is preferable that the deodorant composition according to the embodiment of the present disclosure is produced according to the aspect (2) described above.

The method of producing the specific dispersed particles in the present disclosure is not particularly limited as long as the specific dispersed particles can be produced using the method.

The method of producing copper oxide particles described in JP2016-160124A can be suitably used as the method of producing the specific dispersed particles.

The production method described in JP2016-160124A is a method of producing copper oxide particles by merging a copper salt solution with a basic compound solution for reaction using a flow type reaction.

A flow type reaction system to be used includes introducing a copper salt solution into a first flow path and introducing a basic compound solution into a second flow path so that each solution flows in each flow path; allowing the copper salt solution that flows in the first flow path to be merged with the basic compound solution that flows in the second flow path; and reacting the copper salt with the basic compound during the merged liquid flowing into the downstream to obtain copper oxide fine particles from the reaction product.

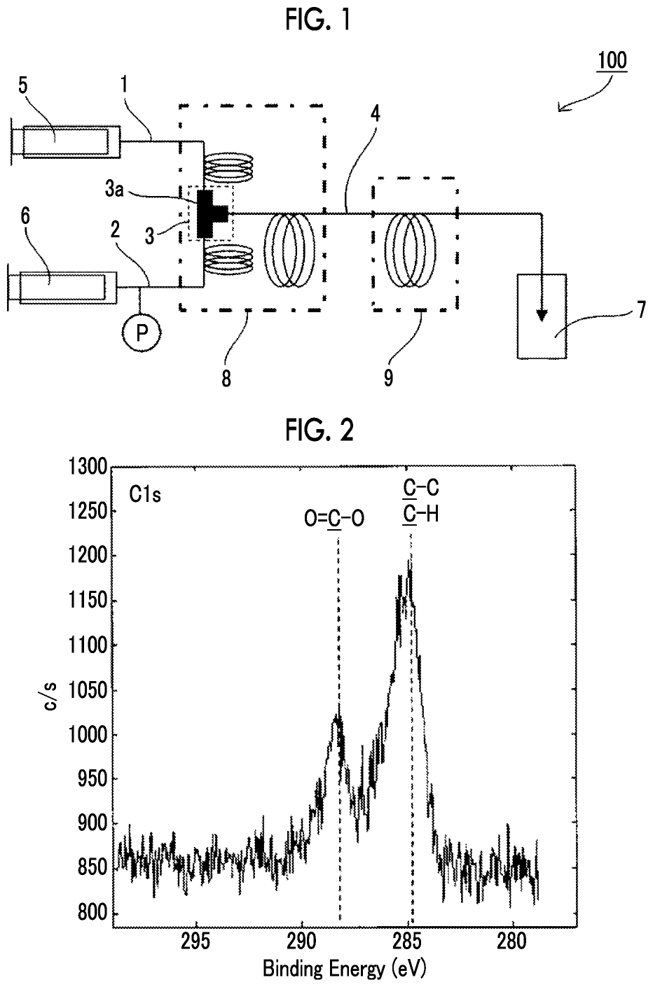

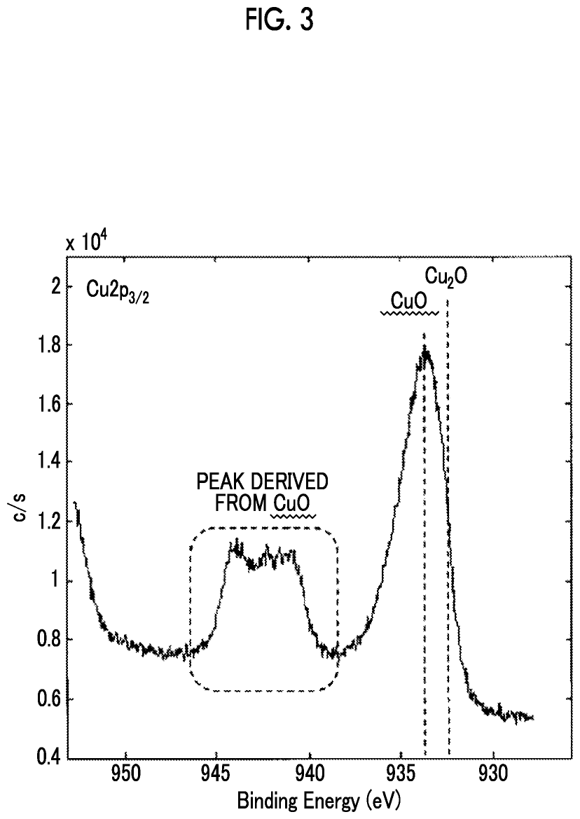

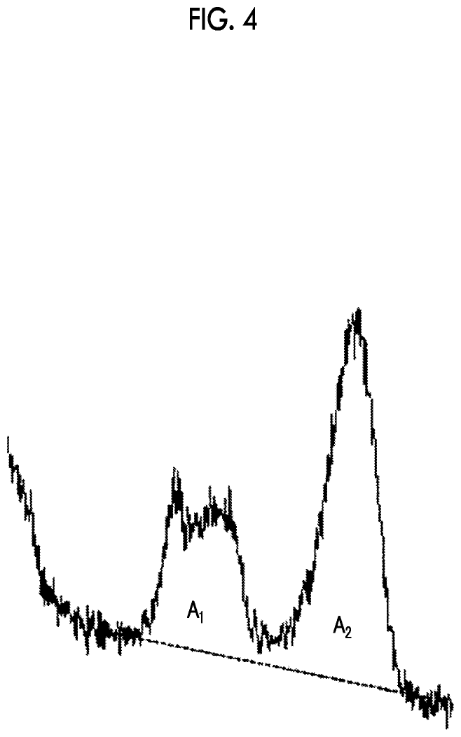

Further, the production method described in JP2016-160124A can be similarly applied to the production of the copper oxide particles A by adjusting various conditions for generating and reacting copper oxide particles, such as the composition and/or the concentration of the copper salt solution and/or the basic compound solution, and the flow rate of the solution at the time of flowing into the flow type reaction system illustrated in FIG. 1, FIG. 3, or FIG. 6 in the same document.

### EXAMPLES

Hereinafter, embodiments of the present invention will be described in more detail based on the following examples, but the deodorant composition according to the embodiment of the present disclosure is not limited to these examples.

[Construction of Flow Type Reaction System]

A flow type reaction system 100 having a configuration illustrated in FIG. 1 was constructed.

In FIG. 1, the reference numeral 1 represents a first flow path, the reference numeral 2 represents a second flow path, the reference numeral 3 represents a merging region, the reference numeral 3a represents T-shaped mixer, the reference numeral 4 represents a reaction flow path, the reference numeral 5 represents copper salt solution introduction means (syringe pump), the reference numeral 6 represents basic compound solution introduction means (syringe pump), the reference numeral 7 represents a recovery container, the reference numeral 8 represents a heating region, the reference numeral 9 represents a cooling region, and P represents a pressure gauge.

As the first flow path (1), the second flow path (2), and the reaction flow path (4), tubes made of SUS316 were used. Syringe pumps (PHD ULTRA, manufactured by HARVARD Apparatus) were used as the copper salt solution introduction means (5) and the basic compound solution introduction means (6), and a configuration in which a syringe (volume of 100 mL) filled with the copper salt aqueous solution and a syringe (volume of 100 mL) filled with the basic compound aqueous solution were respectively mounted on each syringe pump was employed.

The tip of the syringe filled with the copper salt solution was connected to the first flow path having an outer diameter of ⅛ In (3.18 mm) and an inner diameter of 2.17 mm. Further, the tip of the syringe filled with the basic compound solution was connected to the second flow path having an outer diameter of ⅛ In (3.18 mm) and an inner diameter of 2.17 mm. A pressure gauge P was installed in the second flow path 2 so that the pressure inside the flow path at the time of sending a liquid was able to be measured.

In the region on the downstream side in the first flow path 1, a tube having a length of 50 cm, an outer diameter of 1/16 In (1.59 mm), and an inner diameter of 1 mm was wound in a coil shape and disposed in the heating region 8. The heating region 8 was an oil bath in the present example. Further, similar to the region on the downstream side in the second flow path 2, a tube having a length of 50 cm, an outer diameter of 1/16 In (1.59 mm), and an inner diameter of 1 mm was wound in a coil shape and disposed in the heating region 8.

A T-shaped mixer 3a (manufactured by Upchurch Scientific Inc.) having an inner diameter of 0.5 mm was installed at the ends of the first flow path 1 and the second flow path 2 on the downstream side terminal, and the opening portions (A and B) of the T-shaped mixer (trade name: TEE UNION, manufactured by Upchurch Scientific Inc.) were connected to each flow path such that the copper salt solution and the basic compound solution collided with each other. The remaining opening portion O of the T-shaped mixer was connected to a flow path wound in a coil shape and having a length of 2 m, an outer diameter of ⅛ In (3.18 mm), and an inner diameter of 2.17 mm, and this flow path was installed in the heating region (8, water bath (20° C.)). Further, a flow path wound in a coil shape and having a length of 1 m, an outer diameter of ⅛ In (3.18 mm), and an inner diameter of 2.17 mm was connected to the downstream side of the heating region, and this flow path was installed in the cooling region 9. The recovery container 7 was installed on the downstream side of the cooling region 9, and the reaction solution was recovered.

<Production of Dispersed Particles 1 and Dispersion Liquid 1>

A dispersion liquid 1 containing dispersed particles 1 as the specific dispersed particles was prepared in the following manner.

A copper (II) acetate hydrate was dissolved in water and diluted with water to prepare a copper acetate aqueous solution (concentration of 0.285 mol/L).

A 50% (mass/volume) sodium hydroxide aqueous solution was diluted with water to prepare a sodium hydroxide aqueous solution (concentration of 0.399 mol/L).

Each glass syringe (volume of 100 mL) was filled with 100 mL of the copper acetate aqueous solution and 100 mL of the sodium hydroxide aqueous solution and was provided in the syringe pump of the flow type reaction system. Each liquid was sent at 5 mL/min. In this flow type reaction system, the temperature of the heating region (8) was set to 90° C. 100 mL of the liquid (dispersion liquid containing the dispersed particles 1) having passed through the reaction flow path was recovered in the recovery container (a polyethylene container having a volume of 250 ml).

The average primary particle diameter of the obtained dispersed particles 1 (copper oxide particles) was 7 nm.

The obtained dispersion liquid was subjected to ultrafiltration, impurities were removed and concentrated, and the content of the dispersed particles 1 in the dispersion liquid was adjusted to 1.1% by mass to obtain a dispersion liquid 1.

The ultrafiltration was performed by providing an ultrafiltration membrane (molecular weight cutoff of 10000, manufactured by Toyo Roshi Kaisha, Ltd.) for a stirring type ultra-holder (model number: UHP-76K, manufactured by Toyo Roshi Kaisha, Ltd.). The impurities such as ions were removed to obtain a predetermined conductivity by performing filtration during the addition of the same amount of water as the amount of the filtrate flowing out through the filtration.

### Example 1

<Preparation of Specific Metal Salt Solution>

Copper (II) acetate monohydrate was dissolved in water to prepare a copper acetate aqueous solution having a copper ion content of 0.0003% by mass as a specific metal salt solution 1.

<Production of Deodorant Composition>

The specific metal salt solution 1 was added to the dispersion liquid 1 obtained in the above-described manner to prepare a deodorant composition of Example 1 in which the content of the dispersed particles 1 was 0.003% by mass.

### Example 2

<Preparation of Specific Metal Salt Solution 2>

Copper (II) nitrate trihydrate was dissolved in water to prepare a copper nitrate aqueous solution having a copper ion content of 0.0003% by mass as a specific metal salt solution 2.

<Production of Deodorant Composition>

A deodorant composition of Example 2 was prepared in the same manner as in Example 1 except that the dispersion liquid 1 and the specific metal salt solution 2 obtained in the above-described manner were used.

### Example 3

<Preparation of Specific Metal Salt Solution 3>

Zinc acetate dihydrate was dissolved in water to prepare a zinc acetate aqueous solution having a zinc ion content of 0.0003% by mass as a specific metal salt solution 3.

<Production of Deodorant Composition>

A deodorant composition of Example 3 was prepared in the same manner as in Example 1 except that the dispersion liquid 1 and the specific metal salt solution 3 obtained in the above-described manner were used.

### Example 4

<Preparation of Specific Metal Salt Solution 4>

A specific metal salt solution 4 was prepared in the same manner as that for the specific metal salt solution 1 except that the amount of the copper (II) acetate monohydrate to be used was changed and the copper ion content was set to 0.0006% by mass in the preparation of the specific metal salt solution 1.

<Production of Deodorant Composition>

A deodorant composition of Example 4 was prepared in the same manner as in Example 1 except that the dispersion liquid 1 and the specific metal salt solution 4 obtained in the above-described manner were used.

### Example 5

<Preparation of Specific Metal Salt Solution 5>

A specific metal salt solution 5 was prepared in the same manner as that for the specific metal salt solution 2 except that the amount of the copper (II) nitrate trihydrate to be used was changed and the copper ion content was set to 0.0006% by mass in the preparation of the specific metal salt solution 2.

<Production of Deodorant Composition>

A deodorant composition of Example 5 was prepared in the same manner as in Example 1 except that the dispersion liquid 1 and the specific metal salt solution 5 obtained in the above-described manner were used.

### Example 6

<Preparation of Specific Metal Salt Solution 6>

A specific metal salt solution 6 was prepared in the same manner as that for the specific metal salt solution 3 except that the amount of the zinc acetate dihydrate to be used was changed and the zinc ion content was set to 0.0006% by mass in the preparation of the specific metal salt solution 3.

<Production of Deodorant Composition>

A deodorant composition of Example 6 was prepared in the same manner as in Example 1 except that the dispersion liquid 1 and the specific metal salt solution 6 obtained in the above-described manner were used.

### Example 7

<Preparation of Specific Metal Salt Solution 7>

A specific metal salt solution 7 was prepared in the same manner as that for the specific metal salt solution 1 except that the amount of the copper (II) acetate monohydrate to be used was changed and the copper ion content was set to 0.0015% by mass in the preparation of the specific metal salt solution 1.

<Production of Deodorant Composition>

A deodorant composition of Example 7 was prepared in the same manner as in Example 1 except that the dispersion liquid 1 and the specific metal salt solution 7 obtained in the above-described manner were used.

### Example 8

<Preparation of Specific Metal Salt Solution 8>

A specific metal salt solution 8 was prepared in the same manner as that for the specific metal salt solution 2 except that the amount of the copper (II) nitrate trihydrate to be used was changed and the copper ion content was set to 0.0015% by mass in the preparation of the specific metal salt solution 2.

<Production of Deodorant Composition>

A deodorant composition of Example 8 was prepared in the same manner as in Example 1 except that the dispersion liquid 1 and the specific metal salt solution 8 obtained in the above-described manner were used.

### Example 9

<Preparation of Specific Metal Salt Solution 9>

A specific metal salt solution 9 was prepared in the same manner as that for the specific metal salt solution 3 except that the amount of the zinc acetate dihydrate to be used was changed and the zinc ion content was set to 0.0015% by mass in the preparation of the specific metal salt solution 3.

<Production of Deodorant Composition>

A deodorant composition of Example 9 was prepared in the same manner as in Example 1 except that the dispersion liquid 1 and the specific metal salt solution 9 obtained in the above-described manner were used.

### Examples 10, 11, 13, and 14

<Preparation of Alcohol Aqueous Solution>

Each alcohol aqueous solution was prepared by mixing water and alcohol at a ratio at which the content of alcohol in the prepared deodorant composition became the alcohol content described in the column of the “content of alcohol in dispersion medium” in Table 1 using the alcohol described in the column of “alcohols” in Table 1.

<Production of Deodorant Composition>

The alcohol aqueous solution prepared in the above-described manner was added to the dispersion liquid 1 obtained in the above-described manner such that the content of the dispersed particles 1 was set to 0.003% by mass, thereby preparing each deodorant composition of Examples 10, 11, 13, and 14.

### Examples 12 and 15

<Preparation of Alcohol>

The alcohol described in the column of “alcohols” in Table 1 was prepared.

<Production of Deodorant Composition>

The alcohol prepared in the above-described manner was added to the dispersion liquid 1 obtained in the above-described manner such that the content of the dispersed particles 1 was set to 0.003% by mass, thereby preparing each deodorant composition of Examples 12 and 15.

### Example 16

<Preparation of Specific Metal Salt Solution 16>

Each ethanol aqueous solution was prepared by mixing water and ethanol at a ratio at which the content of alcohol in the prepared deodorant composition became the alcohol content described in the column of the “content of alcohol in dispersion medium” in Table 1.

Copper (II) acetate monohydrate was dissolved in the ethanol aqueous solution obtained in the above-described manner to prepare a specific metal salt solution 16 having a copper ion content of 0.0003% by mass.

<Production of Deodorant Composition>

The dispersion liquid 1 obtained in the above-described manner was diluted with the specific metal salt solution 16 such that the content of the dispersed particles 1 was set to 0.003% by mass, thereby preparing a deodorant composition of Example 16.

### Example 17

<Preparation of Specific Metal Salt Solution 17>

Copper (II) acetate monohydrate was dissolved in ethanol to prepare a specific metal salt solution 17.

<Production of Deodorant Composition>

The dispersion liquid 1 obtained in the above-described manner was diluted with the specific metal salt solution 17 such that the content of the dispersed particles 1 was set to 0.003% by mass, thereby preparing a deodorant composition of Example 17.

### Example 18

<Preparation of Specific Metal Salt Solution 18>

An isopropanol aqueous solution was prepared such that the content of isopropanol after addition to the dispersion liquid 1 became the content described in the column of the “content of alcohol in dispersion medium” in Table 1.

Copper (II) acetate monohydrate was dissolved in the isopropanol aqueous solution obtained in the above-described manner to prepare a 0.0003 mass% specific metal salt solution 18.

<Production of Deodorant Composition>

The dispersion liquid 1 obtained in the above-described manner was diluted with the specific metal salt solution 18 such that the content of the dispersed particles 1 was set to 0.003% by mass, thereby preparing a deodorant composition of Example 18.

### Example 19

<Preparation of Specific Metal Salt Solution 19>

Copper (II) acetate monohydrate was dissolved in isopropanol to prepare a specific metal salt solution 19.

<Production of Deodorant Composition>

The dispersion liquid 1 obtained in the above-described manner was diluted with the specific metal salt solution 19 such that the content of the dispersed particles 1 was set to 0.003% by mass, thereby preparing a deodorant composition of Example 19.

### Comparative Example 1

Water was added to the dispersion liquid 1 obtained in the above-described manner such that the content of the dispersed particles 1 was set to 0.003% by mass, thereby preparing a deodorant composition of Comparative Example 1.

[Measurement and Evaluation]

1. Observation and Physical Properties of Dispersed Particles

(Measurement of Average Secondary Particle Diameter)

The average secondary particle diameter (nm) of the dispersed particles 1 was obtained by diluting the dispersion liquid 1 obtained in the above-described manner with water, preparing a sample for measurement whose content was set to 0.01% by mass, and performing measurement according to the following method.

The dynamic light scattering (DLS) average particle diameter in the dispersion liquid 1 obtained by performing the above-described treatment was measured using a dynamic light scattering determination device (ZETASIZER ZS, manufactured by Malvern Instruments Ltd.). The average particle diameter was measured according to a method defined in ISO13321 as the average value (Z-Average) of the particle diameter based on cumulant analysis.

The average secondary particle diameter (nm) of dispersed particles 1 was 40 nm.

(Peak Area Ratio (1))

The dry powder of the dispersed particles 1 was measured under the following conditions according to X-ray photoelectron spectroscopy (XPS). The dried powder of the dispersed particles 1 was obtained by centrifuging 130 mL of the above-described dispersion liquid 1 at approximately 10000 G (1 G=9.80665 m/s²) so that the particles were precipitated and vacuum-drying the obtained paste at 40° C. for 5 hours.

The C1s peak derived from a trace amount of contamination present on the surface was calibrated to 284.8 eV (FIG. 2), and the peak area ratio was calculated by setting the area of a peak derived from divalent CuO present in a range of 938.5 eV to 948 eV as A₁ and the area of a peak derived from all components containing Cu present in a range of 928 eV to 938.5 eV as A₂ based on 938.5 eV in the spectrum of Cu₂p_{3/2} shown in FIG. 3. The range of the peak area A₁ and the range of the peak area A₂ were set as shown in FIG. 4.

The abundance ratio of cuprous oxide in the dispersed particles was acquired from the following calculation formula as the peak area ratio (1).

Peak area ratio (1)=A1/(A1+A2)

As the result, it was confirmed that the dispersed particles 1 were copper oxide particles respectively having a particle surface on which cuprous oxide had been formed based on the fact that the calculated value of the peak area ratio (1) was 0.04.

XPS Measurement Conditions:

X-ray source: monochromatic Al-Kα rays (100 μmϕ, 25 W, 15 kV)

Charge correction: possible (combination of electron gun and slow ion gun)

Photoelectron extraction angle: 45°

Measurement range: 300 μm² (area)

Pass Energy: 23.5 eV

Measurement elements: Cu₂p, Cu LMM, C1s

Energy correction: correction of C1s to 284.8 eV

(Measurement of Zeta Potential)

The dispersion liquid 1 was diluted with water so that the concentration thereof was set to 0.01% by mass. A predetermined amount of the dispersion liquid was introduced into a dedicated measurement cell made of glass, and the zeta potential (mV) was measured using ELS-Z1EAS (manufactured by OTSUKA ELECTRONICS Co., Ltd.).

The zeta potential of the dispersed particles 1 was +39 (mV).

(Measurement of Specific Surface Area)

The specific surface area (m²/g) of the dispersed particles 1 obtained in the above-described manner was acquired under the following conditions.

Pre-treatment: dried under reduced pressure at 40° C. for 40 hours

Measuring device: Quantachrome ChemBET3000

Measuring method: BET one-point method, 30% of nitrogen and helium were used

The specific surface area of the dispersed particles 1 was 160 g (m²/g).

(Observation of Dispersion State)

Each deodorant composition was aged at 25° C. for 3 months, and the dispersion state of the dispersed particles 1 in the aged deodorant composition was visually observed. In all deodorant compositions, aggregation of dispersed particles was not observed.

2. Molar Ratio of Dispersed Particles to Hydroxide Ions

The molar ratio of the dispersed particles to the hydroxide ions in each deodorant composition was acquired based on the above-described calculation method. The results are listed in Table 1.

3. Evaluation of Deodorizing Performance

(1) Hydrogen Sulfide Removal Rate (%)

Samples were prepared by allowing the obtained deodorant compositions to be aged respectively for 1 month, 2 months, and 3 months at 25° C.

Immediately after the preparation, the dispersed particles 1 were filtrated from the respective samples aged for 1 month, 2 months, and 3 months, the dispersed particles 1 were dried, and the hydrogen sulfide removal rate (%) was measured using the obtained dried powder.

The results are listed in Table 1.

The dried powder of the dispersed particles 1 was obtained by centrifuging 130 mL of each deodorant composition at approximately 10000 G (1 G=9.80665 m/s²) so that the particles were precipitated and vacuum-drying the obtained paste at 40° C. for 5 hours.

The H₂S removal rate was acquired by measuring the concentration of H₂S in a Tedlar bag filled with odor gas under the following conditions using the dispersed particles 1 applied to filter paper and performing calculation based on Equation A.

As the filter paper, commercially available cellulose filter paper having a basis weight of 450 g/m² and a thickness of 1.5 mm was used.

H₂S removal rate=(concentration ppm of remaining H₂S)/(concentration ppm of initial H₂S)×100 (Equation A)

Coating amount of dispersed particles 1: 0.06 mg in 100 cm²

Test method, standard: fiber cooperative act, detection tube method

Odor gas type: 20 ppm of hydrogen sulfide

Dilution gas condition: mixed with dry N₂ gas, and humidity control carried out under temperature condition of 20° C. at humidity of 65% for 24 hours or longer (as defined in fiber cooperative act)

Time for exposure to odor gas: 2 hours

Volume of Tedlar bag filled with the odor gas: 3L

(2) Variation Coefficient (%) of Hydrogen Sulfide Removal Rate

An absolute value of the variation coefficient (%) of the hydrogen sulfide removal rate (hereinafter, also simply referred to as a “variation coefficient) was calculated using Equation B based on the results of the hydrogen sulfide removal rate immediately after the preparation of each deodorant composition and the hydrogen sulfide removal rate after aging each deodorant composition for 3 months. The results are listed in Table 1.

Variation coefficient (%) of hydrogen sulfide removal rate=[(hydrogen sulfide removal rate after 3 months)/(hydrogen sulfide removal rate immediately after preparation)/hydrogen sulfide removal rate immediately after preparation]×100 (Equation B)

(3) Suppression of Decrease in Deodorizing Effect at Time of Storage for Long Period of Time

The suppression of a decrease in the deodorizing effect of each deodorant composition at the time of storage for a long period of time was evaluated based on the calculated variation coefficient (%). The evaluation standards are as follows.

Evaluation Standards

A: The variation coefficient was 0% or greater and less than 5%.

B: The variation coefficient was 5% or greater and less than 10%.

C: The variation coefficient was 10% or greater.

In a case where the evaluation level was “A” or “B”, it was determined that a decrease in deodorizing effect of the deodorant composition was satisfactorily suppressed at the time of storage for a long period of time.

The details of respective components used for preparation of the deodorant composition described above are as follows.

Copper (II) acetate monohydrate (manufactured by Wako Pure Chemical Industries, Ltd.)

Copper (II) nitrate trihydrate (manufactured by Wako Pure Chemical Industries, Ltd.)

Zinc acetate dihydrate (manufactured by Wako Pure Chemical Industries, Ltd.)

Ethanol (ethanol (99.5), manufactured by Wako Pure Chemical Industries, Ltd.)

Isopropanol (first glass reagent, manufactured by Wako Pure Chemical Industries, Ltd.)

TABLE 1

| | Content of | | Content of metal | | | | |
|---|---|---|---|---|---|---|---|
| | dispersed | | ions to dispersed | | Alcohol content in | | |
| | particles | | particles | | dispersed medium | Deodorizing performance | |
| | [% by mass] | Metal salts | [% by mass] | Alcohols | [% by mass] | Hydrogen sulfide removal rate [%] | |
| Comparative | 0.003 | | 0 | — | 0 | 50 | 50 |
| Example 1 | | | | | | | |
| Example 1 | 0.003 | Cu(Ac)₂ | 10 | — | 0 | 50 | 49 |
| Example 2 | 0.003 | Cu(NO₃)₂ | 10 | — | 0 | 49 | 49 |
| Example 3 | 0.003 | Zn(Ac)₂ | 10 | — | 0 | 48 | 48 |
| Example 4 | 0.003 | Cu(Ac)₂ | 20 | — | 0 | 53 | 50 |
| Example 5 | 0.003 | Cu(NO₃)₂ | 20 | — | 0 | 52 | 51 |
| Example 6 | 0.003 | Zn(Ac)₂ | 20 | — | 0 | 50 | 49 |
| Example 7 | 0.003 | Cu(Ac)₂ | 50 | — | 0 | 58 | 58 |
| Example 8 | 0.003 | Cu(NO₃)₂ | 50 | — | 0 | 59 | 59 |
| Example 9 | 0.003 | Zn(Ac)₂ | 50 | — | 0 | 59 | 58 |
| Example 10 | 0.003 | | 0 | Ethanol | 20 | 50 | 50 |
| Example 11 | 0.003 | | 0 | Ethanol | 30 | 50 | 52 |
| Example 12 | 0.003 | | 0 | Ethanol | 99 | 50 | 51 |
| Example 13 | 0.003 | | 0 | Isopropanol | 20 | 51 | 50 |
| Example 14 | 0.003 | | 0 | Isopropanol | 30 | 50 | 49 |
| Example 15 | 0.003 | | 0 | Isopropanol | 98 | 51 | 50 |
| Example 16 | 0.003 | Cu(Ac)₂ | 20 | Ethanol | 30 | 50 | 49 |
| Example 17 | 0.003 | Cu(Ac)₂ | 20 | Ethanol | 99 | 51 | 50 |
| Example 18 | 0.003 | Cu(Ac)₂ | 20 | Isopropanol | 30 | 50 | 49 |
| Example 19 | 0.003 | Cu(Ac)₂ | 20 | Isopropanol | 98 | 51 | 50 |

| | Deodorizing performance | | | | |
|---|---|---|---|---|---|
| | | | Suppression of decrease in deodorizing effect at | | |
| | | | time of storage for long period of time | | |
| | | | Variation coefficient of | | Molar ratio of |
| | | | hydrogen sulfide removal rate | | dispersed particles |
| | Hydrogen sulfide removal rate [%] | | [%] | Evaluation | to hydroxide ions |
| Comparative | 41 | 40 | 20 | C | 602 |
| Example 1 | | | | | |
| Example 1 | 48 | 46 | 8 | B | 9545 |
| Example 2 | 49 | 46 | 6 | B | 12017 |
| Example 3 | 48 | 44 | 8 | B | 9545 |
| Example 4 | 47 | 53 | 0 | A | 13099 |
| Example 5 | 49 | 50 | 4 | A | 15893 |
| Example 6 | 50 | 50 | 0 | A | 13119 |
| Example 7 | 57 | 58 | 0 | A | 14089 |
| Example 8 | 59 | 58 | 2 | A | 16102 |
| Example 9 | 57 | 57 | 3 | A | 14543 |
| Example 10 | 48 | 46 | 8 | B | 792 |
| Example 11 | 51 | 50 | 0 | A | 927 |
| Example 12 | 51 | 51 | 2 | A | 60226 |
| Example 13 | 50 | 47 | 8 | B | 792 |
| Example 14 | 51 | 51 | 2 | A | 927 |
| Example 15 | 52 | 50 | 2 | A | 30113 |
| Example 16 | 51 | 51 | 2 | A | 18712 |
| Example 17 | 52 | 50 | 2 | A | 60226 |
| Example 18 | 51 | 51 | 2 | A | 18712 |
| Example 19 | 52 | 50 | 2 | A | 30113 |

In Table 1, “-” in the columns of alcohols indicates that the alcohol was not used as the specific solvent.

In Table 1, Cu(Ac)₂, Cu(NO₃)₂, and Zn(Ac)₂ respectively correspond to copper acetate, copper nitrate, zinc acetate which are specific metal salts used for preparation of the specific metal salt solution.

In Table 1, “-” in the columns of the molar ratio of the dispersed particles to the hydroxide ions indicates that the dispersion medium was formed of only alcohol and the molar ratio of the dispersed particles to the hydroxide ions was not calculated.

As listed in Table 1, in each example, it was found that the variation coefficient of the H₂S removal rate was small and the decrease in the deodorizing effect at the time of storage for a long period of time, for example, 3 months, was satisfactorily suppressed.

Meanwhile, in Comparative Example 1, it was found that the deodorizing effect was as excellent as in the examples in a case of immediately after the preparation and after aging the composition for 1 month, but the deodorizing effect was decreased in a case of storage for a long period of time, for example, 2 months or longer, compared to each example.

The disclosure of JP No. 2017-189852 filed on Sep. 29, 2017 is incorporated herein by reference.

In a case where all documents, patent applications, and technical standards described in the present specification are specified to be incorporated specifically and individually as cited documents, the documents, patent applications, and technical standards are incorporated herein in the same limited scope as the cited documents.

### EXPLANATION OF REFERENCES

100: flow type reaction system

1: first flow path

2: second flow path

3: merging region

3a: T-shaped mixer

4: reaction flow path

5: copper salt solution introduction means (syringe pump)

6: basic compound solution introduction means (syringe pump)

7: recovery container

8: heating region

9: cooling region

P: pressure gauge

What is claimed is:

1. A deodorant composition comprising:
dispersed particles which have an average secondary particle diameter of 200 nm or less and are formed of at least one kind of particles selected from metal particles or metal oxide particles and each of which has a surface that does not contain a dispersant; and
at least one kind selected from an aqueous solvent other than water or a metal salt having a monovalent, a divalent, or a trivalent metal ion.

2. The deodorant composition according to claim 1,
which comprises the metal salt, wherein the metal salt is at least one kind selected from metal salts having divalent metal ions.

3. The deodorant composition according to claim 2,
which comprises the metal salt, wherein the metal salt is at least one kind selected from a copper salt, a zinc salt, or a magnesium salt.

4. The deodorant composition according to claim 1,
which comprises the metal salt, wherein a content of a metal ion derived from the metal salt is in a range of 10% by mass to 50% by mass with respect to a total mass of the dispersed particles contained in the deodorant composition.

5. The deodorant composition according to claim 1,
which comprises the aqueous solvent, wherein the aqueous solvent is an alcohol, and
a content of the alcohol is 20% by mass or greater with respect to a total mass of a dispersion medium contained in the deodorant composition.

6. The deodorant composition according to claim 1,
which comprises the aqueous solvent, wherein the aqueous solvent is a monovalent alcohol having 1 to 3 carbon atoms.

7. The deodorant composition according to claim 1,
wherein a molar ratio of the dispersed particles to hydroxide ions contained in the deodorant composition is 800 or greater.

8. The deodorant composition according to claim 1,
wherein the dispersed particles are copper oxide particles.

9. The deodorant composition according to claim 1,
wherein a content of the dispersed particles is in a range of 0.0001% by mass to 14% by mass with respect to a total mass of the deodorant composition.

10. The deodorant composition according to claim 1, wherein:
the dispersed particles are copper oxide particles;
the deodorant composition comprises the metal salt, and the metal salt is at least one kind selected from a copper salt, a zinc salt, or a magnesium salt;
a content of a metal ion derived from the metal salt is in a range of 10% by mass to 50% by mass with respect to a total mass of the dispersed particles contained in the deodorant composition; and
a molar ratio of the dispersed particles to hydroxide ions contained in the deodorant composition is 800 or greater.

11. The deodorant composition according to claim 1, wherein:
the dispersed particles are copper oxide particles;
the deodorant composition comprises the aqueous solvent, and the aqueous solvent is a monovalent alcohol having 1 to 3 carbon atoms,
the aqueous solvent is an alcohol, and a content of the alcohol is 20% by mass or greater with respect to a total mass of a dispersion medium contained in the deodorant composition; and
a molar ratio of the dispersed particles to hydroxide ions contained in the deodorant composition is 800 or greater.

12. The deodorant composition according to claim 1, wherein:
the dispersed particles are copper oxide particles;
the deodorant composition comprises the aqueous solvent and the aqueous solvent;
the aqueous solvent is a monovalent alcohol having 1 to 3 carbon atom, and a content of the alcohol is 20% by mass or greater with respect to a total mass of a dispersion medium contained in the deodorant composition;
the metal salt is at least one kind selected from a copper salt, a zinc salt, or a magnesium salt, and a content of a metal ion derived from the metal salt is in a range of 10% by mass to 50% by mass with respect to a total mass of the dispersed particles contained in the deodorant composition; and
a molar ratio of the dispersed particles to hydroxide ions contained in the deodorant composition is 800 or greater.

13. The deodorant composition according to claim 1, which is used for deodorizing odor caused by hydrogen sulfide.
